(19) United States
(12) Patent Application Publication
(10) Pub. No.: US 2006/0025704 A1
(43) Pub. Date: Feb. 2, 2006

(54) Device for measuring parameters in the brain

(75) Inventors: Rudiger Stendel, Berlin (DE); Karlheinz Gohler, Zwonitz (DE); Gerd Kunze, Zwonitz (DE); Friedrich Gropp, Berlin (DE)

Correspondence Address: BROWDY AND NEIMARK, P.L.L.C.;624 NINTH STREET, NW, SUITE 300, WASHINGTON, DC, 20001-5303, US

(73) Assignee: RAUMEDIC AG, Helmbrechts (DE)

(21) Appl. No.: 10/526,071

(22) PCT Filed: Aug. 26, 2003
(86) PCT No.: PCT/EP03/09411

§ 371 Date: Feb. 28, 2005

(30) Foreign Application Priority Data
Aug. 29, 2002 (DE) 102 39 743.0

(51) Int. Cl. A61B 5/00; A61M 31/00; A61B 5/05

(52) U.S. Cl. 600/561; 600/353; 600/355; 600/549; 604/93.01

(57) ABSTRACT

The invention relates to an implantable device in modular-system design for measuring brain parameters. It consists of an electronics unit and a sensor unit integrated therein. The electronics unit with the essential components transmitter, receiver, power supply and control unit is sealed solidly and tightly, it is reusable after its sterilization. The sensor unit comprises at least one single lumen catheter and the sensors arranged therein for pressure and/or temperature measurement.

It is known prior art to implant probes epidurally or subdurally for measuring brain pressure or other parameters in the human brain. These probes are equipped with measurement sensors that measure the brain pressure, convert it into electric signals and transmit it over a cable connection to a patient monitor.

There, the measured values are processed and displayed in the form of numerical readings and graphically as curves.

The cable connections between probes having measurement sensors and the patient monitors, however, can be created only with significant effort since the patient monitors have differently designed sockets and are prone to defects in their operation. Additionally, almost every diagnostic measuring element requires a special cable so that especially in the intensive care unit patients are connected to a confusing amount of cables, which results in complications in patient care and presents a risk for the patient.

This applies especially if the patient must be treated in stressful situations or is being transported.

Faulty measurements or the total failure of the measurement probes may be possible, with the consequence that new measurement probes must be implanted.

Overall, the use of cable connections is thus expensive and in particular cases it is associated with high risks for the patient.

Probes of this type with integrated measurement sensors for implantation are produced, for example, by firms REHAU AG÷Co., Johnson & Johnson, Camino, Medtronic.

The subject of DE 43 29 898 A1 is a wireless medical diagnosing and monitoring system, for example also for neuromonitoring. The system comprises an evaluator station and one or more electrodes that are attached to the surface of the patient's skin.

The electrodes comprise a digital transmitting unit with antenna, optionally a receiving unit, a power supply unit, as well as at least one semiconductor sensor. The semiconductor sensors may be used, among other things, for the detection of EEG or EKG signals.

This solution has the shortcoming that only electrodes can be used that are attached to the patient's skin surface.

The attempt to implement, either permanently or on an outpatient basis, brain pressure measurements in shunt systems for the treatment of hydrocephali has produced combinations of implanted measuring probes with sensors whose measuring signals are telemetrically linked to the given evaluator unit.

DE 197 05 474 A1, for example, describes an implantable measuring unit for measurements, among other things, of brain pressures. The sensor element and telemetry unit therein are affixed on a flexible film. The telemetry unit has an external coil whereby the implanted circuit board is powered inductively, additionally the data measured in the transmitter element is inductively transmitted to the evaluator unit.

The shortcoming is that such an inductive wireless transmission of data or power works only across a very short distance—a few millimeters—so that only epidural and possibly also subdural measurements are possible. DE 43 41 903 A1 describes a particularly small, implantable device whose outer dimensions are smaller than 1.0×1.5×0.6 mm and which is suitable for continuous measuring of the pressure and/or flow and/or temperature in bodies or organs of humans or animals. This device transmits network values or measuring signals percutaneously, without cabling system, to a receiver located outside the body that processes the measuring signals and brings them to display.

Such sensor-telemetry-unit systems that are integrated on a chip, i.e., tightly coupled, are not suitable for measuring the desired parameters (for example brain pressure, temperature) at the locations that are optimal for the indication.

The reason is that they can be implanted problem-free only epidurally, as well as possibly also subdurally. Their implantation into the locations that are much more suitable for the measurements, namely into the parenchyma or the ventricles, is not possible.

In these regions the external power supply by means of induction or HF fields is also virtually impossible, as a result of which the measuring and transmitter unit are functional only for a short time.

Additionally, the often necessary additional use of imaging processes, such as magnetic resonance imaging, leads to malfunctions of the implanted control and regulation technology or to inductive currents in the circuit system, and last but not least to the heating of and damage to the tissue surrounding the implants.

In general, it may be stated with respect to the described prior art in connection with the telemetric transmission of signals from implanted sensors, that no reports have been available up to now regarding their successful practical implementation.

Given that, in addition to the design of the sensitive and specific sensors, the measuring locations in particular are crucial for the correct measurement of physiological data in the human brain, the object of the present invention therefore presented itself to provide a device for measuring parameters in the brain that has the following features:
Measuring of the desired parameters is possible at the usual=classic, medically accepted locations, namely in the parenchyma and/or in the ventricles; if required, the epidural or subdural measurements shall remain possible as well.
The transmission and processing of patient data takes place digitally and via telemetry.
A modular system is available whereby the measuring device—depending on the given requirement—can be assembled tailor-made.
The electronics unit is reusable after sterilization thereof.

This object has been met with the invention in such a way that
the sensors are arranged in a catheter of polymeric materials, which optionally incorporates at least one lumen for the drainage of fluid
the electronics unit is received in an enclosed assembly of preferably annular design
the catheter is fixed solidly and tightly but removably in the centrical cutout of the base plate by means of an annular fastening element
the sensor unit and electronics unit are connected to one another by means of a micro plug
the measuring unit with the catheter and sensor, and the electronics unit that is mounted on the base plate with the power supply and above the same a removable cover is placed completely under the scalp on the skull bone and fully enclosed toward the outside.

The invention shall now be explained in more detail below:

The base plate is semi-flexible, it comprises a centrical cutout with connection piece and integrated annular fastening element. Alternatively, a ball housing with a valve may be provided in its place that is suitable for catheters of at least two different sizes and that also permits the slanted seat of the catheter in the base plate.

The base plate, after its completion with the electronics unit and catheter, is provided for implantation purposes with a flexible, tight-fitting and removable cover.

The sensor unit comprises a catheter having one or more sensors for measuring, for example, brain pressure, temperature, CO₂ saturation, or pH, etc. For fluid drainage, at least one lumen may also be integrated in the catheter. The catheter has at its proximal end a micro plug that creates the connection to the electronics unit, so that the measuring signals can be acquired and relayed to the evaluator unit.

The electronics unit that is disposed underneath the semi-flexible cover is resterilizable and thus reusable after disassembly from the base plate and decoupling of the catheter by unplugging of the micro plug.

It is a particular advantage of the inventive device that, due to the modular design, the components can be assembled based on the application at hand. For example, a short catheter with a diameter of CH 3 may be used for the measurement in the parenchyma, a short catheter with a diameter of CH 6 for measurements in the ventricle region with fluid drainage.

In accordance with the invention it is also particularly advantageous that the sensor unit and electronics unit are initially separate from one another.

The catheter containing the sensor/sensors can therefore be placed minimally invasively at the optimal measuring locations, namely the ventricles or the parenchyma, in the usual manner, for example—after opening of the scalp and placement of a bore in the skull bone—by means of a sleeve and mandrin.

The proximal end of the catheter is subsequently tightly screwed with the centrical cutout of the base plate over the fastening element and connected to the electronics unit by means of the micro plug. Lastly, the base plate that has been completed in this manner is tightly connected to a semi-flexible cover and the scalp is reclosed.

The embedding of the sensor and connecting wire into a catheter and into a non-metallic sensor housing prevents heat build-up in the surrounding tissue and dislocation at the measuring location and thus the appearance of artifacts during the measurement and application of the imaging diagnostics, especially in magnetic resonance imaging (MRI).

If rechargeable batteries are used, an inductive thermo-electric or HF-field charging is ensured in this manner. Their function may be protected by means of a shielding of the components or by switching off the sensor unit during the MRI exam.

In the case of catheters that incorporate measurement sensors and a lumen for fluid drainage, a connection piece is integrated on the base plate, which leads the lumen away from the measuring unit, near the patient, and couples it to a catheter that leads into the patient's chest cavity or abdominal cavity. A connection to a shunt valve is possible as well.

Every system assembly can therefore in principle be designed as a closed system.

An interesting inventive option for the power supply for the implanted system is as follows: If the brain pressure is measured in the ventricle, the flow of the fluid can be used for power generation by means of a miniaturized dynamo. To this end, the sensor housing has integrated into it a chamber with an inflow and an outflow opening, between which a turbine with a connected dynamo is mounted.

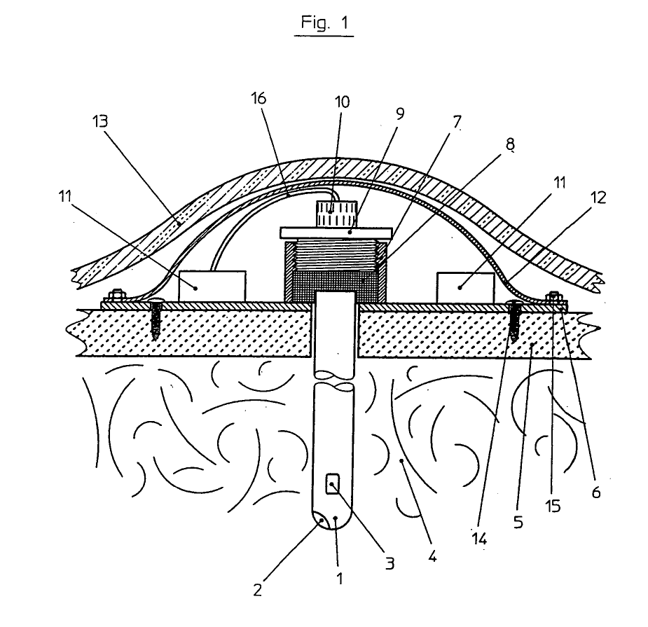

The invention shall now be explained in detail in an example embodiment; in amplification thereof please see the explanatory illustration in FIG. 1.

### EXAMPLE EMBODIMENT

The implanted device in modular-system design consists of a catheter 1, which comprises at its distal end a temperature sensor 2 and a pressure sensor 3 and extends through the skull bone 5 into the brain tissue 4.

The base plate 6 that is fastened on the skull bone 5 by means of a screw 14 comprises an electronics unit and an integrated fastening element 7 with internal thread. By means of this thread the screw 9 exerts a force onto the seal 8 whereby the space between the semi-flexible cover 12 and base plate 6 is closed tightly relative to the brain tissue 4 and at the same time the catheter 1 is secured on the base plate 6 and thus on the skull bone 5.

The micro plug 10 that is located on the proximal end of the catheter 1 is connected via a line 16 to the electronics unit 11.

A radio signal is now used to test the device for functionality. Afterwards the semi-flexible cover 12 is tightly but removably connected to the base plate 6 by means of the screws 15.

The scalp 13 stretches over and protects the implanted device.

1-8. (canceled)

9. Device for measuring brain parameters
having a sensor unit (1-3) that is designed in such a way that it—
is implantable distally minimally invasively in the parenchyma and/or in the ventricles, —
is received proximally in a fastening element (7) that is arranged centered on a base plate (6),
wherein
the sensor unit (1-3) is connected electrically conducting to an electronics unit (11) by means of a micro plug (10),
the measuring device comprises a proximal assembly comprising:—
the fastening element (7), —
the sections of the sensor unit (1-3) received in the fastening element (7), —
the electronics unit (11), —
the micro plug (10),
wherein the proximal assembly (1-3, 7, 10, 11) is implemented such that it is connected solidly and tightly but removably by means of a semi-flexible cover (12) and positionable between the skull bone and scalp.

10. A device according to claim 9, wherein it is subdivided into modules.

11. A device according to claim 9, wherein the electronics unit (11) comprises as main components a power supply, a transmitter, a receiver, a control unit and a micro-plug socket.

12. A device according to claim 9, wherein the sensor unit (1-3) comprises a catheter (1) of polymeric material and at least one sensor (2, 3) for measuring one of the group of brain pressure, temperature, CO₂ partial pressure, oxygen partial pressure.

13. A device according to claim 12, wherein the catheter (1) contains at least one lumen for sensor components, optionally additionally at least one lumen for the drainage of fluid.

14. A device according to claim 12, wherein the catheter (1) that contains a lumen for the fluid drainage is connected by means of a connection piece of the base plate to an additional catheter placed in the patient's abdominal cavity and together with the additional catheter forms a closed system.

15. A device according to claim 9, wherein the electronics unit (11) is sterilizable and reusable.

16. A device according to claim 9, wherein the electronics unit (11) uses endogenous energies through utilization of one of the group of thermal elements or piezoelectronic devices or nanoturbines in the subarachnoid space.
